(19) United States
(12) Patent Application Publication
(10) Pub. No.: US 2008/0107753 A1
(43) Pub. Date: May 8, 2008

(54) Compositions to alleviate menopausal symptoms

(75) Inventor: Jeffrey Wycoff, Boulder, CO (US)

Correspondence Address: CR MILES, P.C.;CRAIG R. MILES, 405 MASON COURT, SUITE 119, FORT COLLINS, CO, 80524, US

(73) Assignee: Paradigm, Inc.

(21) Appl. No.: 11/977,012

(22) Filed: Oct. 22, 2007

Related U.S. Application Data
(60) Provisional application No. 60/853,565, filed on Oct. 23, 2006.

(51) Int. Cl. A61K 35/58; A61P 43/00

(52) U.S. Cl. 424/542

(57) ABSTRACT

Compositions and methods of preparing and using such compositions to treat the symptoms of perimenopause and menopause.

This United States Non-provisional Patent Application claims the benefit of U.S. Provisional Patent Application No. 60/853,565, filed Oct. 23, 2006, hereby incorporated by reference.

### BACKGROUND

Compositions and methods of preparing and using such compositions to treat the symptoms of perimenopause or menopause.

Menopause is conventionally defined as the time in a woman's life when there have been no menstrual periods for twelve consecutive months and no other biological or physiological cause can be identified for the cessation of menstrual periods. The changes of the menopause transition (perimenopause) begin about 6 years before the natural menopause. This is a time when the levels of hormones produced by the aging ovaries fluctuate leading to irregular menstrual patterns (irregularity in the length of the period, the time between periods, and the level of flow).

The onset of perimenopause or menopause can be associated with many symptoms among women, but typically include one or more of the following menopausal symptoms: hot flashes, night sweats, mood swings, forgetfulness, vaginal dryness, fluctuations in sexual desire, numbness, tingling, insomnia, nervousness, depression, vertigo, fatigue, arthralgia, myalgia, headaches, palpitations, formication, achy joints, early waking, frequent urination, among others in varied combinations and permutations.

Perimenopausal and menopausal symptoms are thought to be related primarily to the change in estrogen levels, and estrogen replacement therapy, either with or without progesterone, is a common method of providing some relief from menopausal symptoms.

A significant problem with using conventional hormone replacement therapy to treat perimenopause or menopause can be the adverse effects associated with hormone replacement therapy including, but not limited to, nausea, headache, breast tenderness, vaginal bleeding, weight gain, edema, pre-menstrual like symptoms, depression, irritability, an increased risk of stroke, clots, coronary events, and breast cancer.

Due to the various adverse effects associated with hormone replacement therapy, certain herbal remedies have been utilized to alleviate perimenopausal and menopausal symptoms such as black cohosh, pasque flower, or dong quai, or combinations thereof. Homeopathic remedies have also been utilized to alleviate perimenopausal and menopausal symptoms such as Hyland's Menopause Tablets, which contains a specific combination of Amyl Nitrosum, Sanguinaria Canadensis and Lachesis Mutus in a lactose base and for example similar combinations in combination with Cimicifuga Racemosa as described by U.S. Pat. No. 6,248,307. Other known homeopathic products useful in treating perimenopausal and menopausal symptoms are described in the Homeopathic Pharmacopeia of the United States (HPUS). There are benefits to utilizing herbal and homeopathic remedies because they appear to reduce certain perimenopausal and menopausal symptoms, such as hot flashes, and because the compliance rate can be high while the rate of side effects can be low.

Despite advances in the art of herbal and homeopathic remedies, there remains a need for additional compositions, formulations, mixtures, potency dilution admixtures, or the like and methods of preparing and using such compositions, formulations, potency dilution admixtures, or the like to treat the symptoms of perimenopause and menopause symptoms

### SUMMARY OF THE INVENTION

Accordingly, a broad object of the invention can be to provide compositions, formulations, and potency dilution admixtures for the treatment of perimenopausal or menopausal symptoms.

Another broad object of the invention can be to provide improved methods for preparing formulations, particularly oral liquid and solid dosage forms, useful in treating perimenopausal or menopausal symptoms.

Another broad object of the present invention to provide methods of treating perimenopausal or menopausal symptoms such treatment preventing or providing a reduction in the frequency or severity of at least one such symptom, or both the frequency or severity of at least one such symptom, or otherwise mitigating such symptoms.

Another broad object of the invention can be to provide methods of preparing an oral dosage form for the treatment of perimenopausal and menopausal symptoms. Such dosage forms may be prepared by admixing or combining the requisite amounts of homeopathic or herbal components, or both, together with any pharmaceutical excipients, and dividing the mixture into unit doses containing an appropriate amount of the admixture or combination to treat perimenopausal or menopausal symptoms when administered to a person whether in liquid or solid dosage forms.

Another broad object of the invention can be to provide a method of treating perimenopausal and menopausal symptoms by administering a unit dose of the homeopathic components herein described orally to a person as a treatment for perimenopausal and menopausal symptoms.

Naturally, further objects of the invention are disclosed throughout other areas of the specification, drawings, photographs, and claims.

### A BRIEF DESCRIPTION OF THE DRAWINGS

FIGS. 1-4 each provide examples of particular non-limiting embodiments of the inventive compositions encompassed by the invention.

### DETAILED DESCRIPTION OF THE PREFERRED EMBODIMENTS

Compositions and methods of preparing and using such compositions to treat the symptoms of perimenopause or menopause.

The process of homeopathic drug preparation is based on the concept of potentization or potentiation. Homeopathy relies on the administration of successively more diluted formulations of key homeopathic components to affect a desired response. The manufacturing process underlying this philosophy, therefore, requires the preparation of dilutions that represent very small fractions of the original base product's composition. Products available commercially or prepared by homeopathic physicians and pharmacists may be combined with alcohol, distilled water, or lactose as their dilution matrix. Mother tincture (also referred to as “Ø”) typically refers to a crude homeopathic compound that is triturated in alcohol.

Potency designations refer to the dilution of the mother mix. One part drug mixed with 9 parts dilution matrix is designated a 1× potency. A potency designation of 10× (or 1c) is composed of 1 part mother tincture diluted in 99 parts of a selected diluent. A potency designation 1m is 1 part mother tincture mixed in 999 parts of selected dilution. A potency of 2× is 1 part 1× potency and 9 parts of dilution. Low potency examples are 1×, 6×, 6c. Examples of medium potency include 30× and 30c. High potency examples are 200c, 1m, 20m.

“HPUS” as referred to herein means a material prepared in accordance to the specifications of the Homeopathic Pharmacopoeia of the United States, hereby incorporated by reference herein.

The term “composition” as used herein refers to any single material or combination, mixture, or admixture of materials, or admixture of dilution potencies, or mixture of diluted materials (whether diluted as mixtures of active substances with excipients as solids or diluted as mixtures of active substances with excipients as liquids), or the like, an amount of which can provide a dose, or an amount of which can be divided into a plurality of doses, or can be divided into amounts a plurality of which can be combined to provide a dose (such as two or more capsules, tablets, drops, or measures of a powder, or the like). A “dose” upon administration to a patient affects perimenopausal or menopausal symptoms as above-described. The term “dosage form” as used herein refers to a dose established in any manner capable of delivery to a patient and without limitation includes solid dosage forms such as an amount powder, an amount of effervescent powder, an amount of the composition pressed or compacted to provide a solid typically configured to be swallowed such as a tablet, or a plurality of tablets, or a number of tablets which individually or in combination provide a dose, a caplet, a capsule which contains an amount of the composition, and further includes liquid dosage forms such as an amount of liquid, a drop of liquid, a plurality of drops of liquid, or a number of drops of liquid which individually or in combination allow delivery of a dose. The term oral dosage form whether a liquid oral dosage form or a solid oral dosage form provides delivery of a dose by introduction of the dosage form(s) whether liquid or solid into the mouth. The term “tablet” as used herein refers to an amount of the composition pressed or compacted or otherwise established in a solid form (including without limitation the active substances applied as liquids to pressed or compacted amounts of excipients in solid form) configured to be taken orally.

A dosage form can include a composition which provides in sufficient amount an admixture of Lachesis mutans venom, amyl nitrate; and sulfuric acid for treatment of menopausal symptoms in a patient. Lachesis mutans is a venomous snake commonly known as the Bushmaster. The venom of this snake can be utilized in a dosage form prepared in accordance with the HPUS. Amyl nitrate (amyl nitrosum) is a mixture of the nitrite esters of 3-methyl-1-butanol and 2-methyl-1-butanol. Amyl nitrate contains not less than 85% C.sub.5 H.sub. 11 NO.sub.2. The dosage form can prepared in accordance with HPUS. Sulfuric acid H.sub.2 SO.sub.4 is a strong mineral acid soluble in water at all concentrations. Although 100% sulfuric acid can be made, this loses SO.sub.3 at the boiling point to produce 98.3% acid. A 98% grade is more stable for storage. The dosage form of 98% grade of H.sub.2 SO.sub.4 can be prepared in accordance with the HPUS.

Certain homoeopathic embodiments of the composition can provide Lachesis mutans venom of between about 6×HPUS and about 200c HPUS, amyl nitrate of between about 6×HPUS and about 200c HPUS, and sulfuric acid of between about 3×HPUS and about 200c HPUS. The dosage form can further include at least one component selected from the group consisting of: an amount of folliculinum and an amount of estradiol. Folliculinum, is made from Oestrone, a synthetic form of estrogen. Folliculinum can be utilized in a dosage form prepared in accordance with HPUS. Estradiol (17β-estradiaol)(CAS number 50-28-2) is a female steroid hormone C.sub.18H.sub.24 O.sub.2 which is produced by women during the reproductive years by the granulosa cells of the ovaries by aromatization of testosterone from the theca cells. Certain dosage forms can further include folliculinum of between 3×HPUS and about 200c HPUS with a particular dosage form providing 6c HPUS. Other dosage forms can further include estradiol of between about 6× and about 30c HPUS (or equivalent molar amount) with a particular dosage form providing estradiol of 6c (or equivalent molar amount).

Preferred oral solid dosage forms retain the original HPUS strength of dilution as above described or as set out in Tables 1-4 in tablet dosage form to provide an admixture of Lachesis mutans venom, amyl nitrate, sulfuric acid and in certain embodiments at least one of folliculium or estradiol can be added. As shown in Table 2 as but one non-limiting example, a preferred oral dosage form can comprise two tablets to provide Lachesis mutans about 6c HPUS, Amyl Nitrate about 6×HPUS, Folliculinum about 6c HPUS, and Sulfuric Acid about 12×HPUS. As to this embodiment of the invention, two tablets can be taken orally every three to four hours to effect menopausal symptoms.

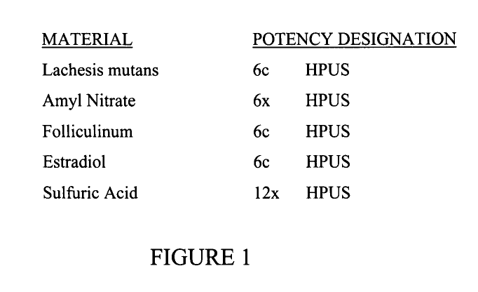

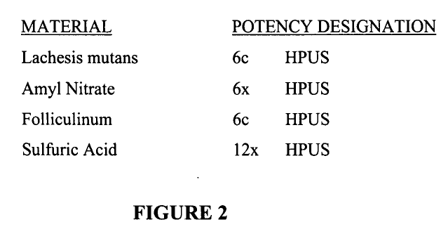

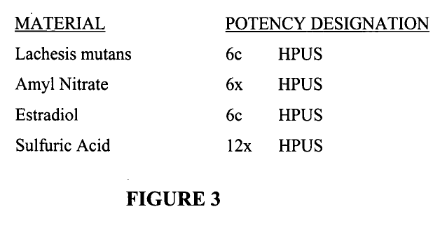

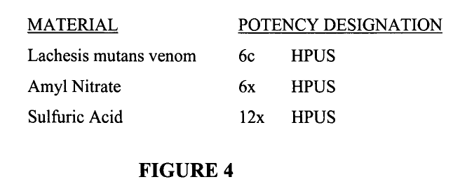

The description of these preferred embodiments of the invention in tablet dosage form is not intended to limit the invention to solid dosage forms as tablets and solid oral dosage forms can further comprise amounts of powders, capsules, or the like as described above which retain the original HPUS strength of dilution as set out in Tables 1-4 and FIGS. 1-4 or which fall in the ranges of dilution above-described. Rather the descriptions include herein are intended to provide examples from which the person of ordinary skill can make and use the numerous and wide variety of dosage forms which can be prepared as various permutations and combinations within the potency ranges referred to including but not limited to solid oral dosage forms but also liquid oral dosage forms which would typically include the HPUS strength of dilution typically in about 2-10 drops administered orally. It is preferred that the dosage form when delivered in drops of a diluent retain the original HPUS strength of dilution.

TABLE 1

| MATERIAL | POTENCY DESIGNATION | |
|---|---|---|
| Lachesis mutans | 6c | HPUS |
| Amyl Nitrate | 6x | HPUS |
| Folliculinum | 6c | HPUS |
| Estradiol | 6c | HPUS |
| Sulfuric Acid | 12x | HPUS |

TABLE 2

| MATERIAL | POTENCY DESIGNATION | |
|---|---|---|
| Lachesis mutans | 6c | HPUS |
| Amyl Nitrate | 6x | HPUS |
| Folliculinum | 6c | HPUS |
| Sulfuric Acid | 12x | HPUS |

TABLE 3

| MATERIAL | POTENCY DESIGNATION | |
|---|---|---|
| Lachesis mutans | 6c | HPUS |
| Amyl Nitrate | 6x | HPUS |
| Estradiol | 6c | HPUS |
| Sulfuric Acid | 12x | HPUS |

TABLE 4

| MATERIAL | POTENCY DESIGNATION | |
|---|---|---|
| Lachesis mutans venom | 6c | HPUS |
| Amyl Nitrate | 6x | HPUS |
| Sulfuric Acid | 12x | HPUS |

A method of preparing a solid oral dosage form as a tablet to provide the potency designations set out in Tables 1-4 or the potency designations set out in the ranges described can for example include Amyl Nitrate, Lachesis Muta, Sulfuric Acid, and Folliculinum mixed in an amount of 87% ethyl alchohol-distilled water (v/v). This mixture can be added to Lactose N.F. and mixed to homogeneity, dried and triturated. An appropriate amount of the powder mixture can be combined with magnesium stearate, microcrystalline cellulose, and sucrose, passed through a 20 mesh screen and pressed into a tablet oral dosage form two of which provide Amyl Nitrate (6×HPUS Dilution), Lachesis Muta (6c HPUS Dilution), Sulfuric Acid (12×HPUS Dilution, and Folliculinum (6c HPUS Dilution).

It is not intended that the examples provided above be limiting with respect to the admixture of additional herbal, homeopathic, or excipient components so long as the additional components do not substantially alter the potency designation of the components as above-listed in Tables 1-4 in a dose regardless of dosage form. As such, the dosage forms of the invention may also contain pharmaceutical excipients such as fillers, binders, colorants, flavorants, or the like. The dosage forms of the present invention are prepared by admixing the components together and dividing the mixture into unit doses (number of drops) of desired strength, preferably such that each unit dose provides an effective amount of the components to provide relief from perimenopausal or menopausal symptoms when administered to a patient. Due to physical limitations, a unit may be subtherapeutic but can be formulated to provide an effective does when administered in multiple, i.e. two or more, unit doses or dosage forms at a time. The unit dose can be encapsulated or prepared as tablets according to conventional techniques. To prepare the finished dosage form, the herbal or homeopathic components may be admixed with pharmaceutical adjuvants and encapsulated or prepared as tablets or otherwise prepared as known in the art.

As can be easily understood from the foregoing, the basic concepts of the present invention may be embodied in a variety of ways. The invention involves numerous and varied compositions to alleviate menopausal symptoms.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “homeopathic drug preparation” should be understood to encompass disclosure of the act of “preparing a homeopathic drug”—whether explicitly discussed or not—and, conversely, were there effectively disclosure of the act of “preparing a homeopathic drug”, such a disclosure should be understood to encompass disclosure of a “homeopathic drug preparation” and even a “means for preparing a homeopathic drug.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the compositions to treat menopausal symptoms herein disclosed and described, ii) the related methods of treating menopausal symptoms disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth below are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

I claim:

1. A dosage form of a homeopathic composition, comprising:
a. an amount of Lachesis mutans venom;
b. an amount of amyl nitrate; and
c. an amount of sulfuric acid.

2. The dosage form of a homeopathic composition as described in claim 1, further comprising at least one component selected from the group consisting of: an amount of folliculinum and an amount of estradiol.

3. The dosage form of a homeopathic composition as described in claim 2, wherein said amount of Lachesis mutans venom comprises an amount of Lachesis mutans venom of between about 6×HPUS and about 200c HPUS, and wherein said amount of amyl nitrate comprises an amount of amyl nitrate of between about 6×HPUS and about 200c HPUS, and wherein said amount of sulfuric acid comprises an amount of sulfuric acid of between about 3×HPUS and about 200c HPUS.

4. The dosage form of a homeopathic composition as described in claim 3, wherein said amount of folliculinum comprises and amount of folliculum of between about 3×HPUS and about 200c HPUS.

5. The dosage form of a homeopathic composition as described in claim 6, wherein said amount of estradiol comprises an amount of estradiol of between about 6×HPUS and about 30c HPUS.

6. The dosage form of a homeopathic composition as described in claim 7, wherein said amount of Lachesis mutans venom of between about 6×HPUS to about 200c HPUS comprises and amount of Lachesis mutans venom of about 6c HPUS, and wherein amount of amyl nitrate of between about 6×HPUS to about 200c HPUS comprises an amount of amyl nitrate of about 6×HPUS, and wherein said amount of sulfuric acid of between about 3×HPUS to about 200c HPUS comprises an amount of sulfuric acid of about 12×HPUS.

7. The dosage form of a homeopathic composition as described in claim 8, wherein said amount of folliculinum of between about 3×HPUS to about 200c HPUS comprises and amount of folliculinum of about 6c HPUS.

10. The dosage form of a homeopathic composition as described in claim 8, wherein said amount of estradiol of between about 6×HPUS to about 30c HPUS comprises an amount of estradiol of about 6c HPUS.

11. The dosage form of a homeopathic composition as described in claim 10, wherein said dosage form comprises an oral liquid dosage form.

12. The dosage form of a homeopathic composition as described in claim 11, wherein said oral liquid dosage form comprises between about two drops and about 10 drops.

13. The dosage form of a homeopathic composition as described in claim 10, where said dosage form comprises an oral solid dosage form.

14. The dosage form of a homeopathic composition as described in claim 10, wherein said oral solid dosage form comprises a tablet dosage form.

15. A method of preparing a dosage form of a homeopathic composition, comprising the steps of admixing a sufficient amount of Lachesis mutans venom, amyl nitrate, and sulfuric acid to establish a mixture and dividing said mixture to provide a plurality of said dosage form.

16. A method of preparing a dosage form of a homeopathic composition as described in claim 15, further comprising the step of admixing a sufficient amount of at least one component selected from the group of: folliculinum and estradiol.

17. A method of preparing a dosage form of a homeopathic composition as described in claim 16, wherein said step of dividing said mixture to provide a plurality of said dosage form further comprises the step of establishing in each of said plurality of said dosage form an amount of Lachesis mutans venom of between about 6×HPUS to about 200c HPUS, an amount of amyl nitrate of between about 6×HPUS to about 200c HPUS, and an amount of sulfuric acid of between about 3×HPUS to about 200c HPUS.

18. A method of preparing a dosage form of a homeopathic composition as described in claim 17, further comprising the step of establishing in each of said plurality of said dosage form an amount of folliculum of between about 3×HPUS and about 200c HPUS.

19. A method of preparing a dosage form of a homeopathic composition as described in claim 18, further comprising the step of establishing in each of said plurality of said dosage form an amount of estradiol of between about 6×HPUS and about 30c HPUS.

20. A method of preparing a dosage form of a homeopathic composition as described in claim 19, wherein said step of dividing said mixture to provide a plurality of said dosage form further comprises the step of establishing in each of said plurality of said dosage form an amount of Lachesis mutans venom of about 6×HPUS, an amount of amyl nitrate of about 3×HPUS, an amount of sulfuric acid of about 3×HPUS.

21. A method of preparing a dosage form of a homeopathic composition as described in claim 17, further comprising the step of establishing in each of said plurality of said dosage form an amount of folliculum of about 6c HPUS.

22. A method of preparing a dosage form of a homeopathic composition as described in claim 18, further comprising the step of establishing in each of said plurality of said dosage form an amount of estradiol of about 6c HPUS.

23. A method of treating menopausal symptoms exhibited in a patient comprising the steps of orally administering to said patient a solid oral dosage form of claim 1.

24. A method of treating menopausal symptoms exhibited in a patient comprising the steps of orally administering to said patient at least two tablets.
